(19) United States
(12) Patent Application Publication
(10) Pub. No.: US 2022/0054074 A1
(43) Pub. Date: Feb. 24, 2022

(54) METHODS AND KITS FOR RAPID SCREENING FOR HYPOSMIA AND ANOSMIA

(71) Applicant: University of Rochester, Rochester, NY (US)

(72) Inventors: Christopher J STODGELL, Rochester, NY (US); Ronald W. WOOD, Rochester, NY (US)

(73) Assignee: University of Rochester, Rochester, NY (US)

(21) Appl. No.: 17/410,949

(22) Filed: Aug. 24, 2021

Related U.S. Application Data
(60) Provisional application No. 63/069,440, filed on Aug. 24, 2020.

(51) Int. Cl. A61B 5/00; A61B 5/01; A61J 1/06; A61J 1/03; A61K 8/34

(52) U.S. Cl. CPC A61B 5/4011; A61B 5/01; A61K 8/34; A61J 1/035; A61J 1/06

(57) ABSTRACT

The invention provides methods and kits for rapid and inexpensive detection of hyposmia or anosmia, diminishment or loss of the sense of smell. Sudden onset of diminishment or loss of the sense of smell is an early sign of infection with SARS-CoV-2, the causative agent of COVID-19; detecting the sudden onset of diminishment or loss of the sense of smell can therefore be used to rapidly screen a population for persons who should undergo diagnostic testing for a SARS-CoV-2 infection, and more generally to screen persons for olfactory disorders.

### CROSS-REFERENCE TO RELATED APPLICATIONS

This application claims priority to and the benefit of U.S. Provisional Patent Application No. 63/069,440, filed Aug. 24, 2020, the contents of which are incorporated herein by reference for all purposes.

### STATEMENT OF FEDERAL FUNDING

Not applicable.

### PARTIES TO JOINT RESEARCH AGREEMENT

Not applicable.

### BACKGROUND OF THE INVENTION

The SARS-CoV-2 virus is the causative agent of the disease known as COVID-19. In a short time, the COVID-19 pandemic disrupted the world economy, causing hundreds of thousands of deaths, and cancellation of in-person teaching at schools and universities around the world and, in particular, in the U.S. To allow students to return to the classroom and people to return to work, it would be useful to have a rapid and inexpensive screen for persons who may have a SARS-CoV-2 infection. Unfortunately, the main diagnostic methods to detect the virus require acquisition of samples from the patient and subjecting the samples to RT-PCR to detect whether the samples contain SARS-CoV-2 viral DNA. Patients typically wait two to three days for test results. Moreover, the need for testing can overwhelm the available supply of testing material and lead to limitations on to whom such tests will be administered. The limits on the number of tests available, the delays in obtaining results for persons who have been able to undergo molecular diagnostic testing, and the cost of testing, limit the utility of molecular diagnostic tests as a rapid screening and diagnostic mechanism.

Rapid antigen tests detect viral proteins. They are less sensitive than molecular diagnostic tests, but can produce results in less than an hour at equipped sites at a lower price point. All such tests require viral replication to occur to such an extent that sample concentrations are detectable, a period during which the contagion can spread.

There is currently no cost-effective way to provide regularly even a rough screen for persons who might be infected other than taking the temperature of individuals at entrances to buildings or other checkpoints. Taking body temperature with an infrared scanner can detect infection-associated fever, but has proven to have little utility. An arbitrary threshold temperature results in false positive and negative findings that can be attributed to age-related variations between individuals in body temperature, the effects of ambient temperature, exercise, and equipment inaccuracy

Diminution or loss of the sense of smell is known to occur in a large percentage of persons with early-stage SARS-CoV-2 infection. Unfortunately, the standard ways of measuring diminution or loss of sense of smell, such as “Sniffin Sticks” (see, e.g, Hummel et al., Chem Senses 22: 39-52, 1997) are either expensive or are not intended to be used for testing persons who may have a disease that can be spread by contact. Furthermore, single use devices, such as “The Pocket Smell Test,™” (Sensonics Intl., Haddon Heights, N.J.), use “scratch-and-sniff” methods that provide variable amounts of odorant depending on the area of the surface coating that is “scratched off” by the user and on the force applied by the user, and at a cost that is still too high for regular use on large numbers of individuals. These tests rely on inaccurate identification of odorants, and can misidentify those with parosmia, a distorted sense of smell, as having hyposmia or anosmia.

It would be desirable to have methods and devices that allow the rapid and inexpensive screening of a population of persons, such as students, to discriminate between those who are likely to have an active SARS-CoV-2 infection and therefore should undergo diagnostic testing, and those that are not. Surprisingly, the present invention fulfills these and other needs.

### BRIEF SUMMARY OF INVENTION

The invention provides methods and kits for determining rapidly whether a subject has anosmia or hyposmia, or is normosensitive (has a normal sense of smell).

In a first group of embodiments, the invention provides methods for determining rapidly whether a subject has anosmia and, optionally, whether said subject has hyposmia. The methods comprise: (a) providing the subject with a set of a closed first container and a closed second container, wherein the first container contains a first solution consisting essentially of an odorant at a first selected concentration of the odorant and a carrier and the second container contains a second solution which second solution consists essentially of the odorant at a second selected concentration of the odorant, which selected second concentration is expected to be detectable by all normosensitive individuals, and the carrier, and further wherein the first container and the second container are either (i) attached to each other, (ii) encapsulated within a common cover member permeable to odors and which is both unreactive with, and which does not comprise a material reactive with, the first solution or the second solution, or (iii) are blisters on a blister pack, and the first and the second containers do not mix when the first and the second containers are opened, (b) having the subject (i) open the closed first container, thereby releasing into ambient air around the opened container any odor from the odorant in the first solution in the opened container, and (ii) smell the ambient air around the opened container, allowing the subject to determine if the subject can smell the odorant in the ambient air and, (iii) repeat steps (b)(i) and (b)(ii) for the second container, wherein an inability of the subject to smell the odorant in the second container indicates said subject has anosmia. In some embodiments, the set of the closed first container and the closed second container further comprises a third container, which third container is (i) attached to one or both of the closed first container and the second container, (ii) is encapsulated within a common cover member with the closed first container and said second container, or (iii) is a blister on a blister pack with the closed first container and the second container, wherein the closed third container contains a solution consisting essentially of the carrier without an odorant. In some embodiments, the first selected concentration of the odorant in the first solution is zero. In some embodiments, the first selected concentration of the odorant in the first solution is 1 z-score unit below the mean minimum detectable concentration that people with normal olfaction can smell. In some embodiments, the second selected concentration of said odorant in the second solution is a suprathreshold concentration that does not stimulate a trigeminal nerve of a human. In some embodiments, the first selected concentration of the odorant in the first solution is 1 z-score unit below the mean minimum detectable concentration that people with normal olfaction can smell and wherein the second selected concentration of the odorant in the second solution is a suprathreshold concentration that does not stimulate a trigeminal nerve of individuals in the population, wherein detection by the subject of said odorant in the second solution but not in the first solution indicates that the individual has hyposmia. In some embodiments, the first selected concentration of the odorant in the first solution is 1 z-score unit below the mean minimum detectable concentration that people with normal olfaction can smell and wherein the second selected concentration of the odorant in the second solution is a suprathreshold concentration that does not stimulate a trigeminal nerve of individuals in the population, wherein a failure by the individual to detect the odorant both in the first solution and in the second solution indicates that the individual has anosmia. In some embodiments, the first selected concentration of the odorant in the first solution is 1 z-score unit below the mean minimum detectable concentration that people with normal olfaction can smell and wherein the second selected concentration of said odorant in the second solution is a suprathreshold concentration that does not stimulate a trigeminal nerve of individuals in the population, wherein detection by the individual of the odorant in both the first solution and in the second solution indicates that the individual does not have hyposmia or anosmia. In some embodiments, the odorant is n-butanol, and is present in the first container at a concentration of 0.32%, ±one half log unit. In some embodiments, the odorant is n-butanol, and is present in the first container at a concentration of 0.32%. In some embodiments, the odorant is n-butanol, and is present in the second container at a concentration of 1.8%, ±0.2%, to 50%, preferably 1.8%, ±0.2%, to 18%. In some embodiments, the n-butanol is present in the second container at a concentration of 1.8%, ±0.2%, to 5.6%. In some embodiments, the n-butanol is present in the second container at a concentration of 3.2%. In some embodiments, the said odorant is n-butanol, and is present in the first container at a concentration of 0.32%±one half log unit and is present in the second container at a concentration of 1.8%, ±0.2%, to 5.6%. In some embodiments, the odorant is n-butanol, and is present in the first container at a concentration of 0.32%, and is present in second container at a concentration of 3.2%, ±0.5%. In some embodiments, the odorant is phenyl ethyl alcohol (“PEA”), and is present in the first container at a concentration of 0.32%, ±one half log unit. In some embodiments, the PEA is present in the first container at a concentration of 0.32%, ±0.2%. In some embodiments, the PEA is present in the second container at a concentration of about 1.8%, ±0.2%, to 18%. In some embodiments, the PEA is present in the second container at a concentration of 1.8%, ±0.2%, to 5.6%. In some embodiments, the PEA is present in the second container at a concentration of 3.2%, ±0.5%. In some embodiments, the PEA is present in the first container at a concentration of 0.32%, ±one half log unit, and is present in the second container at a concentration of 3.2%, ±0.5%. In some embodiments, the PEA is present in said first container at a concentration of 0.32% and is present in the second container at a concentration of 3.2%. In some embodiments, there is a third closed container, which third container contains a solution consisting essentially of carrier without odorant. In some embodiments, the first container, the second container, or both, are coded by color, numbering, lettering, or a combination of these. In some embodiments, the closed first container, the closed second container, or both, are ampoules. In some embodiments, the ampoules are encapsulated within a cover member. In some embodiments, the closed first container, the closed second container, and the closed third container are ampoules. In some embodiments, the three ampoules are encapsulated within a cover member. In some embodiments, the cover member is not impregnated with a compound reactive with the first solution or with the second solution. In some embodiments, the closed first container, the closed second container, or both, are blisters on a blister pack. In some embodiments, the closed first container, the closed second container, and the closed third container are blisters on a blister pack. In some embodiments, the set of closed containers consists of a closed first container, a closed second container, and a closed third container. In some embodiments, the closed first container, said closed second container, or both, are applicators which release their contents by being crushed. In some embodiments, the closed first container, the closed second container, or both, are impermeable packets. In some embodiments, the subject reports her or her determination to a tester or electronically. In some embodiments, the report is by an oral statement to a tester. In some embodiments, the report is by making an electronic report on a computer or a mobile device. In some embodiments, the subject is being screened for possible infection by SARS-CoV-2. In some embodiments, if said subject does not smell the odorant in the second container, the subject undergoes further testing for infection by SARS-CoV-2. In some embodiments, if the subject does not smell the odorant in the second container, the subject undergoes further testing for diseases or conditions other than infection by SARS-CoV-2 that affect sense of smell. In some embodiments, the disease or condition other than infection by SARS-CoV-2 that affect sense of smell is a virus other than SARS-CoV-2, a bacterial infection, a fungal infection, an environmental agent, a genetic anomaly, a congenital defect, or an injury.

In a second group of embodiments, the invention provides kits for determining rapidly whether a subject has hyposmia or anosmia. The kits consist of: a first container and a second container, wherein the first container contains a first solution consisting essentially of a selected odorant at a first concentration and a selected carrier, and the second container contains a second solution which second solution consists essentially of the odorant at a second concentration and the selected carrier, further wherein (a) the first container and the second container are (i) attached to each other, (ii) encapsulated within a common cover member permeable to odors and which is both unreactive with, and which does not comprise a material reactive with, the first solution or the second solution, or (iii) are blisters on a blister pack, and (b) the first and second solutions in the first and the second containers do not mix when the containers are opened. In some embodiments, the first container, the second container, or both, is an ampoule. In some embodiments, the ampoule is, or the ampoules are, encapsulated within a cover member. In some embodiments, the first container and the second container are ampoules. In some embodiments, the ampoules are separated by an impermeable barrier or area. In some embodiments, the closed first container, the closed second container, or both, are blisters on a blister pack or are containers contained within blisters on a blister pack. In some embodiments, the kits further comprise a closed third container, which closed third container contains a third solution, which third solution consists essentially of the selected carrier without an odorant. In some embodiments, the first selected concentration of said odorant in said first solution is 1 z-score unit above the mean, 1-tailed, determined in a population of indiv iduals with normal olfaction to be detectable by the population of individuals with normal olfaction and wherein the second selected concentration of the odorant in said second solution is a suprathreshold concentration that does not stimulate a trigeminal nerve of individuals with normal olfaction. In some embodiments, the odorant is n-butanol, and is present in the first container at a concentration of 0.32%±one half log unit. In some embodiments, the n-butanol is present in the first container at a concentration of 0.32%±0.5%, to 5.6%. In some embodiments, the odorant is n-butanol, and is present in the second container at a concentration of about 1.8%±0.2%, to 50%, preferably 1.8%±0.2%, to 18%. In some embodiments, the n-butanol is present in the second container at a concentration of 3.2%±one half log unit. In some embodiments, the n-butanol is present in the second container at a concentration of 3.2%. In some embodiments, the odorant is n-butanol, and is present in the first container at a concentration of 0.32%±one log unit and is present in the second container at a concentration of 1.8%±0.2% to 18%. In some embodiments, the n-butanol is present in the first container at a concentration of 0.32% and is present in the second container at a concentration of 3.2%. In some embodiments, the odorant is phenyl ethyl alcohol (“PEA”), and is present in the first container at a concentration of 0.32%, ±one half log unit. In some embodiments, the PEA is present in the first container at a concentration of 0.32%. In some embodiments, the PEA is present in the second container at a concentration of 1.8%±0.2% to 18%. In some embodiments, the PEA is present in the second container at a concentration of 3.2%±one half log unit. In some embodiments, the PEA is present in the second container at a concentration of 3.2%. In some embodiments, the PEA is present in the first container at a concentration of 0.32%, ±one half log unit and is present in the second container at a concentration of 3.2%±one half log unit. In some embodiments, the PEA is present in the first container at a concentration of 0.32% and is present in the second container at a concentration of 3.2%. In some embodiments, the kit further comprises a third closed container, which third container contains a solution consisting essentially of carrier without an odorant.

### BRIEF DESCRIPTION OF THE DRAWINGS

FIG. 1. FIG. 1 is a schematic depicting an exemplary olfaction test kit of two ampoules. The ampoule on the left, labeled “Hyposmia solution,” contains an odorant at a low concentration selected to be easily detected by persons with normal olfaction, but not persons with reduced or compromised olfaction. The ampoule on the right, labeled “Anosmia solution,” contains the same odorant, at a concentration that is expected to make the presence of the odorant detectable by all persons with normal olfaction. In the embodiment depicted, the two solutions in the ampoules are colored with different food colorings to allow the user to know which ampoule to crush and smell first. In the embodiment shown, the ampoules are separated by an impermeable barrier and the distal end of the ampoules have absorbent applicator tips.

FIG. 2. FIG. 2 is a photograph showing an exemplar olfaction test kit of the invention. Two ampoules, one holding an exemplar odorant, n-butanol, at a low concentration and one ampoule holding the same odorant at a higher concentration, have been placed end to end, and the sealed covering of one end of one ampoule and the sealed covering of one end of the second ampoule have been overlapped. A 1.5″ section of black polyolefin heat shrink tubing has then been shrunk into place to hold the two ampoules together to form a kit for testing for hyposmia and anosmia.

FIG. 3. FIG. 3 is a photograph showing an exemplar olfaction test kit of the invention. As in FIG. 2, two ampoules, one holding an exemplar odorant, n-butanol, at a low concentration and one ampoule holding the same odorant at a higher concentration, have been placed end to end, and the sealed covering of one end of one ampoule and the sealed covering of one end of the second ampoule have been overlapped. In this exemplar kit, a 2.0″ section of clear polyolefin heat shrink tubing has been shrunk into place to hold the two ampoules together as a kit for testing for hyposmia and anosmia. The exterior end of the ampoule holding the low concentration of odorant has been lightly dyed yellow to allow it to be readily identified.

### DETAILED DESCRIPTION

As noted in the Background, the SARS-CoV-2 pandemic has caused a global economic downturn, upending lives and infecting millions of individuals in the United States alone. The infectivity of the virus has required the introduction of physical distancing between individuals who are not living together, more stringent cleaning regimens for public places, and disrupted the ability of groups to gather together, while at the same time, limitations on the number of molecular diagnostic tests available, and their cost, has made regular screening of large numbers of individuals impractical. An inexpensive means of detecting individuals with a SARS-CoV-2 infection would be desirable.

While fever, cough, and myalgia are complaints that are not specific to COVID-19, diminution or loss of the sense of smell has emerged as a common feature of SARS-CoV-2 infection. Lechien, J. R., et al. (Annals Int Med, 2020, doi.org/10.7326/M20-2428) studied a population of over 2000 patients with a SARS-CoV-2 infection confirmed by RT-PCR. Of this population, 87% reported loss of sense of smell, making determination of loss of sense of smell a means of capturing more than 80% of persons with early stages of SARS-CoV-2 infection (more than half the patients reporting loss of the sense of smell regained olfactory function within 14 days of loss onset). In contrast, only some 3.2% of adults over 40 are reported to have a loss of sense of smell, and even rarer among young adults. See, e.g., Hoffman, et al., Reviews in endocrine & metabolic disorders, 2016. 17(2): p. 221-240. While loss of the sense of smell can accompany a head trauma, loss of the sense of smell in the absence of head trauma or other obvious explanation provides a strong indication that an individual may have a SARS-CoV-2 infection when viral infections are prevalent.

Unfortunately, current methods and kits for determining olfactory loss are inappropriate for screening populations of individuals for a disease that can be spread by surface contact. Commercially available kits, such as “Sniffin Sticks®” (Burghardt®, Wedel, Germany), provide accurate determinations, but are expensive and intended for handling by multiple persons being tested over a period of time that would necessitate disinfection between each user. Furthermore, people with impaired sense of smell not resulting from SARS-CoV-2 infection may be denied proper evaluation because device reuse is inappropriate during periods of pandemic risk. “Scratch and sniff” cards with a few common odors, such as “The Pocket Smell Test™” (Sensonics Intl., Haddon Heights, N.J.) are less expensive (though still too expensive for wide-scale screening), but may not clearly differentiate persons with hyposmia or anosmia from those a normal sense of smell (normosmia), as they do not control the area scratched, and therefore exposed, by the patient and therefore do not control the amount of odorant to which the patient is exposed. While they can identify a failure to discriminate odorants correctly, they cannot be used for threshold evaluation.

Surprisingly, the present invention provides methods and kits that render it possible to detect the majority of persons with an early stage of SARS-CoV-2 infection, quickly and at low cost. These advantages make it feasible to provide daily screening of populations of individuals, such as individuals preparing to enter a place where people congregate in groups, including schools, university lecture halls, movie theaters, or shopping malls. As reported in the Examples, below, studies conducted with exemplar embodiments of the inventive kits, during the course of the SARS-CoV-2 pandemic to associate the olfactory status of patients at a respiratory clinic against SARS-CoV-2 infection, as determined by reverse transcriptase-polymerase chain reaction (“RT-PCR”) showed that the inventive test kits and methods could be used to detect COVID-19-associated anosmia in a clinical setting.

Olfactory disorders also arise from causes other than SARS-CoV-2 infections, such as injuries, infections, congenital defects, allergies, genetic disorders or the use of medications. Such patients require evaluation of their olfactory function even when pandemics pose a risk of infection from reusable devices. The present invention reduces the risk that persons not already infected with SARS-CoV-2 or another pathogen will become infected by cross-contamination during testing for olfactory function.

In embodiments of the invention, single-use containers containing a fluid are used to provide rapid and inexpensive testing for both hyposmia and anosmia or, in some embodiments, anosmia alone. In these embodiments, two containers are provided. The first container contains a selected carrier fluid containing a selected odorant, which is present at a concentration selected to be high enough to be detected when inhaled through the nose by essentially all persons with a normal sense of smell, but not by persons with hyposmia. The second container contains the same selected odorant in the same selected carrier fluid, but with the odorant at a concentration has been selected to be high enough to be detected by all persons with a normal sense of smell, but still low enough so that it does not activate the trigeminal nerve and produce irritation or a sensation of pain (if using odorants that produce those sensations. To reduce handling of the containers and the potential for further contact spreading of the coronavirus, in preferred embodiments the single-use containers are either (a) attached to one another, (b) disposed in a cover member, such as a sleeve that encapsulates the two containers, (c) are separate blisters in a blister pack. The attached containers can be, for example, ampoules, as described in some of the exemplar embodiments below, or paired, attached packages of moist towelettes encased in a foil, plastic, or other impermeable package, such as the packaging conventionally used to hold moist towelette hand sanitizer wipes. In the latter embodiments, the first towelette packaged in the pair is moistened by a solution holding the low concentration of odorant, while the second is moistened by a solution holding the higher concentration of odorant. In blister pack embodiments, the blisters can contain the fluids containing the odorants, or can contain paired ampoules containing odorant which is released into the air when the ampoules are crushed, or containers with lids which are then removed to allow

At least one of the containers or a material covering a container, is preferably marked, for example, by letters, numbers, color markings, or a combination of these, so that the subject (if the test is being self-administered) or the tester (if the test is being conducted by someone testing the subject's sense of smell) can distinguish the first container from the second. For example, the container with the lower concentration of odorant may be labeled “1” and the second labelled “2.” In some embodiments, the two containers holding the odorants may be provided in a box or other packaging sized to contain and containing, for example, two single-use ampoules, or two moist towelettes, allowing the subject to withdraw from the box the first ampoule or towelette, expose and smell its contents, report whether or not they can smell the odorant, and then withdraw from the box the second ampoule or towelette and repeat the procedure. In some embodiments, the containers may be within a blister or blisters in a blister pack, wherein opening the blister provides access to the container. For example, the blister pack may contain two marked ampoules which hold, respectively, the low and higher concentrations of odorant described above.

The subject is directed to first open the container containing the lower concentration of the odorant, raise the container to the nose, and to smell the air near the container. Once the subject reports being able to smell (or not) the odor of the fluid in the first container, the subject opens the second container, containing the higher concentration of the odorant, smells the odorant released from that container, and reports being able to smell (or not) the odor from that container.

The possible outcomes for testing with the two containers, and their interpretation, may be described by Table 1:

TABLE 1

| | | Low Concentration Ampoule Report | |
|---|---|---|---|
| | | (Can you smell this?) | |
| | | No | Yes |
| High | No | Anosmic | Retest |
| Concentration | Yes | Hyposmic | Normal |
| Ampoule | | | |
| Report | | | |

If the subject is unable to smell the odorant in the first container, but can smell the odorant in the second container, the subject is considered to have hyposmia. If the subject is unable to smell the odorant in either container, the subject is considered to have anosmia. If the subject can smell the odorant in the first (low odorant concentration) container, but cannot smell the odorant in the second (higher odorant concentration) container, that indicates there may be a problem with the set of containers used or their order of evaluation, and the test should be repeated with a fresh set of containers. If the subject can smell the odorant in both containers, the subject is considered to have a normal sense of smell.

Persons with hyposmia or anosmia and who have not previously been diagnosed with a disorder causing either hyposmia or anosmia should be considered as possibly having an early stage of SARS-CoV-2 infection and should not enter the school, lecture hall, or other public facility at which the smell testing is being conducted. Further, such persons should quarantine themselves and undergo definitive diagnostic testing for viral infection or for other conditions that can result in a loss of olfactory sensation.

In some embodiments, the kits and methods are designed to provide a binary answer as to whether the subject has anosmia (such embodiments are sometimes referred to below as “binary tests”. The tests referred to in the preceding paragraph, in contrast, are sometimes referred to herein as hyposmia/anosmia tests.) In these embodiments, two containers are provided. The first container contains a selected carrier fluid without an odorant (in this embodiment, the fluid content of the first container is similar to that of the first container described above, but with the concentration of odorant at zero). The second container contains a selected odorant in the same selected carrier fluid, with the odorant present at a concentration which has been determined to be high enough to be detectable when smelled by essentially 100% of persons with a normal sense of smell, but low enough so that it does not activate the trigeminal nerve, if the odorant has that property. As with the containers in the embodiments discussed above, to reduce the potential for contact spreading of the coronavirus, in preferred embodiments the containers are either (a) attached to one another, (b) disposed in a cover member, such as a sleeve that encapsulates the two containers, or (c) are separate blisters in a blister pack. The containers are preferably marked, such as by letters, numbers, color markings, or a combination of these, so that the subject can be directed to first open the container containing the carrier, but not odorant, and to smell the air immediately over that container (it is understood that the subject will be requested to raise the container to his or her nose before smelling the air over the opened container). Once the subject reports whether he or she has been able to smell anything in the first container, or not, the subject opens the second container, containing the odorant, and smells the air immediately over that container. If the subject is unable to smell the odorant in the second container, the subject has anosmia and could be instructed not enter the school, lecture hall, or other public facility at or outside of which the smell test is being conducted. Persons unable to smell the odorant in the binary test could also be instructed to quarantine themselves until viral status is determined or evaluated for conditions other than a SARS-CoV-2 infection that can result in a loss of olfactory sensation.

In some preferred embodiments, the containers used in the binary tests or the hyposmia/anosmia tests are ampoules, that are “opened” by crushing or squeezing them, depending on the design of the ampoule, to release the contents into the ambient air. In some embodiments, the two containers may be provided in a box sized to contain and containing two single-use ampoules, but no more, allowing the subject to withdraw from the box the first ampoule, expose and smell its contents, and then withdraw from the box the second ampoule and repeat the procedure.

The use of single-use containers is advantageous, as it reduces or removes the possibility that an infected individual will leave virus on the container to infect another user. It is noted that Sniffin Sticks™ and other pen-type smell tests currently used to test the sense of smell are not “single-use containers” within the meaning of the present disclosure. Sniffin Sticks™ and similar products are both provided as kits costing hundreds to thousands of dollars and are priced and are intended for use by multiple subjects over a period of time, a disadvantage during a viral pandemic. And, while it may be true that any item, even an expensive kit, can be discarded after a single use, this is impractical with Sniffin Sticks™ and similar products both due to the cost of the kit and to the waste of material. In contrast, it is expected that embodiments of the present invention comprising two ampoules enclosed, but separated, in a box or sleeve, can be provided for under a dollar, making the cost in materials for testing hundreds of persons for anosmia no greater than the cost of a single Sniffin Sticks™ test kit.

### Odorants

Testing of the ability of individuals to smell odors is well known in the art and scores of compounds or mixtures of compounds providing odors for testing are both well-known and commercially available. The Sniffin Sticks® kits mentioned above, for example, were developed in 1997, as discussed in Hummel et al., Chemical Senses, 22(1):39-52 (1997), doi.org/10.1093/chemse/22.1.39, originally as kits as containing 16 odorants producing common smells. More recently, additional odorants were added providing 16 more smells, for a total of 32. See, Rumeau, Eur Annals Otorhinolaryngology, Head and Neck Diseases, 133(3):203-206 (2016) doi.org/10.1016/j.anor1.2015.08.004; see also, Wolfensberger, Acta Oto-Laryngologica, 120:2, 303-306 (2000), DOI: 10.1080/000164800750001134. The University of Pennsylvania Smell Identification Test, or “UPSIT,” employs odorants producing the following odors: Menthol, Soap, Turpentine, Lemon, Bubble gum, Cinnamon Mint, Cherry, Orange, Chocolate, Lilac, Coconut, Garlic, Smoke, Fruit punch, Watermelon, Rose, Pine, Wintergreen, Paint thinner, Cheddar cheese, Banana, Pizza, Onion, Strawberry, Clove, Gasoline, Grape, Motor oil, Peanut, Grass, Root beer, Lime, Natural gas, Dill pickle, Chili, Black pepper, Musk, Pineapple, Cedar, Gingerbread, Whiskey, Leather, Peach, Apple, Tomato, Skunk, Honey, and Pumpkin pie. See, e.g., Doty et al., Physiology & Behavior, 32: 489-502 (1984).

Scores of other odorants and scents are known. It is known in the art that the ability of individuals to recognize odors can vary among individuals from different cultural backgrounds, as different cultures, for example, differ in the spices used in preparing food. While that can be a potential concern for tests of an individual's ability to identify a particular smell, it is not expected to be a concern for the inventive methods and kits, as the methods and kits are directed to whether the subject can determine that an odor is present, not whether they can identify the odor. It is assumed that a person of skill in the art is familiar with the odorants currently available and can select a particular odorant for use in different embodiments of the inventive methods and kits.

In preferred embodiments, the odorant used in the methods and kits is n-butanol or phenyl ethyl alcohol (“PEA”). These odorants have been widely studied in smell tests and their properties, including concentrations at or above which n-butanol activate the trigeminal nerve, are well-known.

### Odorant Concentrations

Zernecke et al., Rhinology, 48(3):368-373 (2010) (hereafter “Zernecke et al.”), studied the threshold levels at which a sample of 100 normosmic, healthy individuals were able to detect, separately, the presence of n-butanol and PEA. Zernecke et al. tested a series of dilutions of each odorant on the group, as shown in Table 2. Tests using n-butanol started at a concentration of 4%. The data presented in Zernecke et al. allowed us to select a suprathreshold odorant concentration for the second container of n-butanol and for the second container of PEA that only ˜1:2000 “normal” individuals could not smell, and a lower concentration, for the first container in the hyposmia/anosmia test, that was estimated to be detected by 84.13% (1 z-score unit above the mean, 1-tailed) of people with normal olfaction. The two concentrations allow identifying both hyposmic and anosmic persons, based on normal distribution criteria. A 3.2% concentration would miss ˜1 person in 2000, and that person would be severely hyposmic in any circumstance. A one z-score unit is a reasonable initial selection criterion for hyposmia, as approximately 1 in 8 Americans over age 40 (up to 13.3 million people, or 12.4 percent of the population) is reported to have measurable smell dysfunction when tested by “scratch and sniff” testing, with the rate of erroneous identification as the definition of hyposmia and anosmia. Hoffman, et al., Rev Endocr Metab Disord 17, 221-240 (2016); Rawal et al., Chemical Senses, 41(1):69-76 (2016). This 12.4% corresponds to a z-score of 1.155 (one tailed). The hyposmia threshold can be adjusted to the population proportion as desired by the practitioner based on the normal distribution function, and then interpolated from the dilution series in Zernecke et al. It is anticipated that the practitioner can select suitable concentrations of any particular odorant the practitioner may wish to use by following the procedure set forth in Zernecke et al. of recruiting 100 normosmic, healthy individuals and using a series of dilution steps to determine suprathreshold and one z-score unit for the selected odorant.

In some embodiments, the concentration of n-butanol for the first container is 0.32%, ±one half log unit (we choose to bracket the range of concentrations by a half log unit, as the concentrations of the dilution steps in Zernecke et al. are equally spaced on a logarithmic scale. This range roughly equates to multiples of a dilution step in Zernecke et al.), and in some embodiments is 0.32%. In some embodiments, the odorant is n-butanol, and is present in the second container at a concentration of about 1.8% to 50%, about 1.8% to 40%, about 1.8% to 30%, about 1.8% to 20%, about 1.8% to 19%, about 1.8% to 18%, about 1.8% to 17% about 1.8% to 16%, about 1.8% to 15%, about 1.8% to 14%, about 1.8% to 13%, about 1.8% to 12%, about 1.8% to 11%, about 1.8% to 10%, about 1.8% to 9%, about 1.8% to 8%, about 1.8% to 7%, about 1.8% to 6%, about 1.8% to 5%, or about 1.8% to 4%, with “about” in this sentence meaning±0.2%. In some embodiments, the n-butanol is present in the second container at a concentration of 2 to 6%. In some embodiments, n-butanol is present in the second container at a concentration of 3.2 to 5.6%. In some embodiments, the n-butanol is present in said second container at a concentration of 3.2%. In some embodiments, the n-butanol is present in the first container at a concentration of 0.32%±one half log unit, and is present in the second container at a concentration of about 3%-4%, with “about” in this sentence meaning±0.5%. In some embodiments, the odorant is n-butanol, and is present in the first container at a concentration of 0.32% and is present in the second container at a concentration of 3.2%.

It is noted that Zernecke et al. found 4% n-butanol to be a suprathreshold level at which all persons with normal olfaction could smell the n-butanol. Thus, while we have stated higher concentrations above, as they are expected to be detected by all persons with normal olfaction, there is no scientific reason n-butanol would need to be used in the inventive olfaction tests at a concentration higher than 5.6%. N-butanol is, however, inexpensive and non-toxic in the inventive methods, given the limited quantities needed and very limited time of exposure. Accordingly, concentrations of n-butanol higher than 5.6%, such as the 50%, 40%, 30% and 20% concentrations noted above, could be used if for some reason the practitioner wished to do so. It is noted that, if a subject found any particular concentration of odorant unpleasant, they would simply turn away from the container, as subjects routinely do with the ammonia inhalants commonly used to prevent or treat fainting.

In some embodiments, the odorant is phenyl ethyl alcohol (“PEA”). As with n-butanol, the concentration of PEA for the first container is 0.32%, ±one half log unit. In some embodiments, the concentration of PEA in the second container is about 1.8% to 18%, about 1.8% to 17% about 1.8% to 16%, about 1.8% to 15%, about 1.8% to 14%, about 1.8% to 13%, about 1.8% to 12%, about 1.8% to 11%, about 1.8% to 10%, about 1.8% to 9%, about 1.8% to 8%, about 1.8% to 7%, about 1.8% to 6%, about 1.8% to 5%, or about 1.8% to 4%, with “about” in this sentence meaning±0.2%. In some embodiments, the PEA is present in the second container at a concentration of 2 to 6%. In some embodiments, the PEA is present in the second container at a concentration of 3.2 to 5.6%. In some embodiments, the PEA is present in said second container at a concentration of 3.2%. In some embodiments, the PEA is present in the first container at a concentration of 0.32%±one half log unit, and is present in the second container at a concentration of about 3%-4%, with “about” in this sentence meaning±0.5%. In some embodiments, the odorant is PEA, and is present in the first container at a concentration of 0.32% and is present in the second container at a concentration of 3.2%. As with n-butanol, 5.6% PEA is expected to be detectable by all persons with normal olfaction. PEA is currently expensive, and it is therefore not expected that practitioners would have a reason to use it at concentrations higher than 18%.

TABLE 2

| DILUTION STEP | PERCENT CONCENTRATION |
|---|---|
| (ZERNECKE ET AL.) | N-BUTANOL OR PEA |
| 1 | 4 |
| 2 | 2 |
| 3 | 1 |
| 4 | 0.5 |
| 5 | 0.25 |
| 6 | 0.125 |
| 7 | 0.0625 |
| 8 | 0.03125 |
| 9 | 0.01562 |
| 10 | 0.007812 |
| 11 | 0.003906 |
| 12 | 0.001953 |
| 13 | 0.0009765 |
| 14 | 0.0004882 |
| 15 | 0.0002441 |
| 16 | 0.000122 |

The Zernecke et al study was performed on normal subjects with a mean age in their mid-twenties and can be relied on for determining a suprathreshold concentration.

In some embodiments, food coloring or other nontoxic water-soluble color is included with the odorant to indicate the device is ready for use. If clear or translucent containers are used, different colors may be used to provide a tint to the solutions in the containers to readily distinguish them during manufacturing or use. Preferably, the colors chosen can be discriminated by those with the most common forms of colorblindness. Blue vs yellow, red, orange or brown are combinations commonly used for this purpose (the number of persons who have trouble distinguishing blue from yellow are estimated to be less than 1 in 10,0000). To avoid any color-distinguishing problems, a simple light gray can be used to mark one concentration and none for the other, thereby distinguishing the container with one concentration from the other. Preferably, a light tint is used to minimize transfer of color to surfaces that may come in contact with the containers after use. Alternatively, or in addition, the two containers can be distinguished by numbering or lettering them or placing a color on the outside of one of the containers, with or without a second color on the outside of the other container.

### Carriers

Odorants used in embodiments of the invention are typically provided in a carrier fluid, sometimes also referred to herein as a “vehicle.” For n-butanol, the carrier can be “aqua conservata,” which is demineralized water with preservatives. The carrier used for PEA is typically propylene glycol. These carriers are preferred embodiments for use with the odorants mentioned. Carriers may be used without preservatives, if prepared under sterile conditions, or if the odorants themselves are bacteriostatic. It is expected that the practitioner is familiar with the various carriers that have been used with various odorants, and with means of preserving them if necessary, and can select carriers suitable for use with any particular odorant selected for use in a particular embodiment of the inventive methods or kits.

### Ampoules

In some preferred embodiments, the containers are ampoules, which contain the odorants in unit doses at the concentrations selected by the practitioner. Depending on the design of the particular ampoule, the fluid inside the container can be exposed to the ambient air by crushing or squeezing the ampoule or, for ampoules provided with a neck, by breaking off the neck. In preferred embodiments, the ampoule is one designed to place its fluid contents in contact with ambient air by crushing or squeezing the ampoule. It will be noted that some other containers that can be used in embodiments of the inventive methods and kits are opened by removing a lid, while with ampoules, the fluid contents are placed in contact with air around the container by breaking the container. For ease of reference, the manner in which the fluid contents of the containers are placed in contact with ambient air around the container is generally referred to herein as “opening” the container, even where, like some ampoules, the container is crushed to expose the contents to the air.

### Attached Containers

To reduce the number of points of contact in providing subjects with the containers holding the odorant/carrier solutions, the containers are provided in sets. In preferred embodiments, the containers are attached. For example, solutions of two concentrations of an odorant in a carrier, or of a carrier and a concentration of an odorant in a carrier, may be disposed in containers which are held together by a bridge connecting them, as with some of the cases used for storing contact lenses. Containers with a length longer than their width can be attached along their long axis, far enough apart to allow the user to open each independently of the other (this arrangement is sometimes referred to herein as being “parallel.”) Alternatively, the containers can be attached along their short axis (this arrangement is sometimes referred to herein as being connected in “series.”) In some embodiments, the containers are ampoules. An impermeable barrier or other functional separation between the ampoules prevents the fluids in the ampoules from mixing when the ampoules are crushed, squeezed, or otherwise opened. The ampoules may be attached in parallel or in series. When in series, the ampoules are typically connected by being partially encapsulated in a cover member, such as a sleeve fitting around the area in which the two ampoules come together. The cover member should itself be inert with respect to the carrier, odorants, or other chemicals present in the ampoules and should not be impregnated with chemicals that will react with the carrier, odorants, or other chemicals present in the ampoules. If the ampoules are connected in parallel, the ends to which the fluid will flow when the ampoules are crushed are preferably disposed opposite each other to allow the subject to smell the odorant in the container presented and not the one to which it is attached. After the user crushes the ampoule containing the lower concentration odorant (or carrier without odorant, in a binary test), the ampoules are rotated 180°, thereby positioning the opened first container away from the user's nose before the second ampoule is crushed.

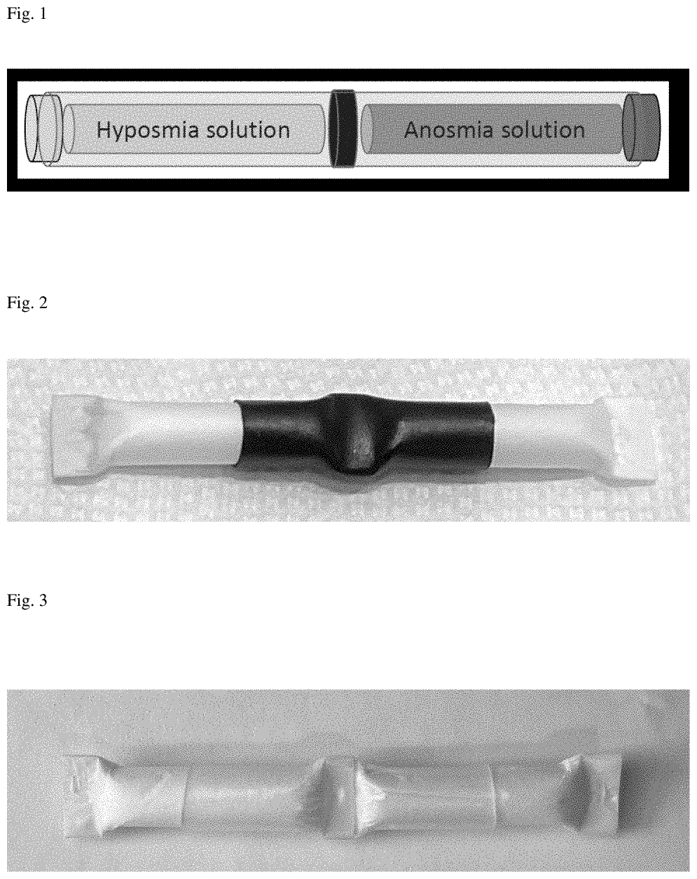

FIG. 1 depicts a schematic diagram of an embodiment of a kit consisting of two ampoules connected in series by an external flexible plastic tube. The left ampoule is labeled “Hyposmia solution,” and contains a low concentration of odorant, while the right ampoule, labeled “Anosmia solution,” contains a higher concentration of odorant that all persons with a normal sense of smell are expected to be able to smell. The two ampoules in the embodiment shown are separated by an impermeable barrier (black ring) and have an absorbent tip (typically of foam or fiber) disposed at the end distal to the impermeable barrier (such devices are sometimes referred to as “crush applicators” and are further described below). When an ampoule is crushed, the fluid contents are able to access and be absorbed into the absorbent tip, thereby gaining exposure to air around the tip. In use, the user is then instructed to raise the tip to the nose and to inhale through the nose to smell the contents and to report whether or not the user can smell the contents.

FIG. 2 is a photograph showing an exemplar olfaction test kit of the invention. Two ampoules, one holding an exemplar odorant, n-butanol, at a low concentration and one ampoule holding the same odorant at a higher concentration that all persons with a normal sense of smell are expected to be able to smell, have been placed end to end, and the sealed cellulose sheath covering of one end of one ampoule and the sealed cellulose sheath covering of one end of the second ampoule have been overlapped. A 1.5″ section of black polyolefin heat shrink wrap has then been shrunk into place to hold the two ampoules together to form the kit. The distal end of the ampoule containing the low concentration odorant has been dyed yellow to allow the user to distinguish which ampoule is which.

FIG. 3 is a photograph showing an exemplar olfaction test kit of the invention. As in FIG. 2, two ampoules, one holding an exemplar odorant, n-butanol, at a low concentration and one ampoule holding the same odorant at a higher concentration that all persons with a normal sense of smell are expected to be able to smell, have been placed end to end, and the sealed covering of one end of one ampoule and the sealed covering of one end of the second ampoule have been overlapped. In this exemplar kit, a 2.0″ section of clear polyolefin heat shrink tubing has been shrunk into place to hold the two ampoules together as a kit. The distal end of the ampoule containing the low concentration odorant has been dyed yellow to allow the user to distinguish which ampoule is which.

In some embodiments, as in FIG. 1, the containers may be “crush applicators.” Crush applicators typically have a crushable, porous foam or fiber end and non-porous surrounds so that, when the crushable end is crushed, a fluid within the applicator can be spread on the skin or other surface, or the end simply smelled. In some embodiments, the crush applicator may have a crushable side with a porous surround, or partial coverage with an impermeable surround. In embodiments of the inventive methods and kits employing attached crush applicators, it is contemplated that the user or tester crushes the crushable end or crushable side of the first crush applicator, which contains just carrier fluid (in the binary anosmia embodiments) or carrier and the low concentration of odorant (in the hyposmia/anosmia embodiments), whereupon the subject smells the tip, or the crushed side, respectively, of the first crush applicator. This procedure is then repeated for the second crush applicator so the subject can smell the crushed end or side, respectively, of the second crush applicator.

In some embodiments, absorbent tips, such as foam or fiber, are disposed at the left end and at the right end of the crush applicator, respectively. The absorbent tips allow solution released from the left-side ampoule and the right-side ampoule, respectively, to wick or flow through the tip, exposing any odorant in the solution to the air around the respective tip. To use, the user crushes the first crush applicator, releasing the solution in the ampoule on the left side of the applicator, and smells the tip to see if the user can detect any odorant wicked or flowed through the fiber tip. The user then repeats the procedure with the second crush applicator.

Embodiments using ampoules typically contain the ampoules within a protective sleeve or sheath (in these embodiments, the sleeve or sheath serves as the cover member). The protective sleeve may be used as a surface on which to label or mark the two sides and helps protect the user from any shards of the underlying tube when the user crushes it. In the embodiments depicted in FIGS. 2 and 3, the sleeves or sheaths are of cellulose and absorb the solution released from the ampoule, allowing the solution within the ampoule contact with ambient air. In the embodiments shown in FIGS. 2 and 3, a portion of the ampoules are further covered by shrink wrap, which both holds the two ampoules together and which serves to further protect the user's fingers.

In early prototypes, we thought that when using ampoules, it would be important to have an impermeable barrier between the ampoules to keep the solution in the first, lower concentration, ampoule from intermingling with the solution in the second, higher concentration, ampoule when the second ampoule was crushed. Some experimentation revealed that an impermeable barrier was not important, as neither the concentration of odorant nor the volume of solution in the first ampoule was sufficient to affect the ability of a user with normal olfaction to smell the solution in the second ampoule. Referring to FIG. 3, which shows the ends of the cellulose sheaths covering the two ampoules overlapped and then held tightly together by shrink wrap, any solution infiltrating from the first ampoule to the second made no difference in use.

In some embodiments, such as having students screen themselves at the entrance of campus buildings, the user can open the first container (such as by crushing an ampoule or pulling the sealing layer of a blister pack, smell the solution contained therein, and then repeat the process for the second container. In the studies reported in the Examples, in which exemplar kits of the type shown in FIG. 3 were used to test patients in a clinic setting, the staff was in full personal protective equipment and disinfected their hands between patients. In that setting, it was found to be convenient for a staff person to administer the smell test by crushing the first ampoule, holding it at arm's length under the nose of the patient, have the patient report if they could smell the first, lower concentration solution, and repeat with the second ampoule.

The crushable applicator embodiments are also useful in embodiments when the test is being administered by a tester to a subject, such as a child or a person with limited mobility in their fingers, who may not have the dexterity to be able to administer a smell test to himself or herself. Advantageously, in some embodiments, crush applicators with impermeable containers or an impermeable cover, such as a shrink-wrapped section, and a permeable end are used that allow a tester administering the smell test to a subject to hold a portion of the applicator that does not get crushed; the tester thereby avoids being wetted with odorants while administering the tests.

In some embodiments, the containers are blisters on blister packs. In embodiments of the invention, each blister pack consists of just two blisters, with the contents as described earlier for the “binary anosmia” and “hyposmia/anosmia” tests. The blisters are preferably marked to allow the user to distinguish between the two blisters. For example, a color may be applied to the exterior of the blisters to allow identifying which blister is which. If the blisters are formed of a clear or a translucent material, the difference in color can be due to tints added to the fluid contents, as explained elsewhere herein.

In some embodiments, the containers are provided as two attached, impermeable packets, typically lined by a foil, similar to those used for packaging alcohol wipes or hand-cleaner wipes (for purposes of this disclosure, the phrase “impermeable packets” refers to these alcohol-wipe like packets). As with the other sets of containers described above, the first packet contains just carrier fluid (in the binary anosmia embodiments) or carrier and a selected low concentration of odorant (in the hyposmia/anosmia embodiments), while the second packet contains a selected higher concentration of odorant for detecting whether the user has anosmia or normal olfaction. The user tears open the top of the first packet, smells the contents, and then, after a brief pause to allow the nose to clear any odorant in the first packet, opens and smells the contents (such as a moist towelette) of the second packet.

### Odorants

As noted, in the inventive methods, the containers holding the carrier or carrier plus odorant are opened and smelled by the subject. The containers may be accompanied by written instructions (such as a sign posted near an open box or bin containing kits for students to use before entering a university building) or by oral instructions for the subject to place the freshly opened container 2-3 cm under the nostrils and to inhale the air above the container deeply through the nose.

### Disposal Containers

In some embodiments, the kits are provided with a further, disposal container for receiving the containers holding the odorants once they have been used. To reduce the build-up of odorant around the disposal container, the disposal container may have one or more features that reduce leakage of odor from the disposal container. For example, the disposal container may have an air-tight lid sealing odors in except for the limited period used kits are being placed in the container or being removed for safe disposal, may hold some activated charcoal, baking soda, calcium chloride or absorbent material, or may have a layer of water to cover the containers holding odorant so that the odorant dissolves into the water rather than dispersing into the air around the disposal container. (For clarity, the term “container” as used in this disclosure refers to a container holding odorant, carrier, or both, unless modified with an adjective, such as “disposal” container.

### Reporting

In some embodiments, the subject reports on whether he or she can smell odorant in the first container, second container, or both the first and the second container. In some embodiments, the subject makes the report orally to a tester. In some embodiments, the subject makes the report electronically, by clicking on a button or link on a computer, tablet, or mobile device.

### EXAMPLES

### Example 1

This Example describes exemplar kits for conducting smell tests that were used in the study reported in Example 2.

Exemplar test kits were developed using sterile, sealed, crushable borosilicate glass ampoules sheathed in a cellulose acetate absorbent layer with a paper coating heat sealed at each end. (James Alexander Corp., Blairstown N.J.). The ampoules were filled with a solution containing either a low concentration of an exemplar odorant, n-butanol, or a higher concentration of that odorant. The odorant solutions were colored with yellow food coloring (low n-butanol concentration) or blue food coloring (higher n-butanol concentration) One end of the paper coating the ampoules containing the low concentration of n-butanol was then marked with the corresponding color for ease in distinguishing the two ampoules after they were assembled into kits.

The ampoules were assembled into kits of two ampoules, one holding n-butanol at a low concentration and one ampoule holding it at a higher concentration, joined end to end. Initially, the two ampoules were joined by overlapping the sealed covering of one end of one ampoule and the sealed covering of one end of the second ampoule and then sliding over them a 1.5″ section of black polyolefin heat shrink tubing that was then shrunk into place. The exterior-facing end of the ampoule holding the low concentration of odorant was dyed yellow to allow ready identification of which ampoule is which. FIG. 2 is a photograph showing an assembled kit of this type.

Our second generation of kits employed a 2.0″ length of clear polyolefin heat shrink tubing to join the ampoules together. The longer shrink tubing extended to cover the point at which users tend to crush the ampoules, thus preventing potential, but rare, contact with glass shards from the ampoule. The clear tubing was chosen to enhance the ability to see the food coloring from the solution in each ampoule once they were crushed. The two ampoules for each kit were placed together with one set of ends overlapping (with the colored end of the low concentration ampoule placed to the outside for easy visibility), the shrink wrap was placed over them, and the assemblage was placed in an oven and heated at 240° F. for ten minutes to shrink the tubing, thereby holding the two ampoules together. The exterior-facing end of the ampoule holding the low concentration of odorant was dyed yellow to allow ready identification of which ampoule is which. FIG. 3 is a photograph showing an assembled kit of this type.

### Example 2

This Example reports the results of using exemplar kits of the invention to screen patients for possible Covid-19 infections in a clinic setting.

Smell kits of the invention were incorporated as an intake procedure to the University of Rochester Primary Care Central Respiratory Clinic, an outpatient clinic dedicated for care of patients with respiratory complaints at risk of COVID-19. Smell test results were recorded in the medical record system in the interval of observation from 14 Dec. 2020 to 7 Jan. 2021, during which 225 olfactory tests were conducted that had accompanying RT-PCR results. The mean age of the patients was 54.3, standard deviation 16.2; median age was 55 for both males and females.

Chi Square analysis. The purpose of this test is to determine if a difference between observed and expected data is due to chance. Viral infection was associated with highly significant effects when the frequency of anosmia was compared to those patients with normal function (χ2_{(Idf)}=9.97, p=0.0016). There were no sex differences observed in viral positivity (χ2_{(Idf)}=1.95, p=0.163).

Odds ratios and relative risk. Odds ratios provide a measure of the strength of association between exposure and an outcome, representing the odds that an outcome will occur in the presence of SARS-CoV-2 exposure compared to those in the absence of viral infection. The odds ratio (OR), its standard error (SE), and the 95% confidence interval and are calculated according to Altman (PRACTICAL STATISTICS FOR MEDICAL RESEARCH. 1991: Chapman & Hall/CRC, Boca Raton, Fla.). The standard normal deviate (z-value) is ln (OR)/SE (ln (OR)). Relative risk is the ratio of the probability of an outcome in the two groups.

The results of the testing showed the results set forth in Table 3.

TABLE 3
Odds ratio

| | | | | 95% confidence | | |
|---|---|---|---|---|---|---|
| | Odds ratio | SE | Z | limits | | p |
| Anosmia | 3.36 | 0.394 | 3.07 | 7.26 | 1.55 | 0.0021 |

Relative risk ratio

| | Relative | | | 95% confidence | | |
|---|---|---|---|---|---|---|
| | risk | SE | Z | limits | | p |
| Anosmia | 2.40 | 0.279 | 3.15 | 4.15 | 1.39 | 0.0016 |

The risk of anosmia was 2.4 times greater if a patient displayed SARS-CoV-2 positivity (p=0.0016, two tailed).

### Example 3

This Example reports the age-adjusted estimate of the expected proportion of patients in the study reported in Example 2 that exhibited olfactory dysfunction compared to the number that would be expected in a normal healthy population.

The National Health and Nutrition Examination Survey (“NHANES”) estimated the prevalence of olfactory dysfunction in the U.S. population over the age of 40, providing estimated proportions in ten-year age brackets. (Hoffman, H. J., et al., Reviews in endocrine & metabolic disorders, 2016. 17(2):221-240). The NHANES survey information was used to provide an estimated proportion likely to show olfactory dysfunction (“OD”) based on the age distribution of the patients, as reported in the previous Example.

The observed and expected proportions are shown in Table 4.

TABLE 4
Observed proportions of OD in respiratory clinic sample

| SARS-CoV-2 positive | | SARS-CoV-2 negative | |
|---|---|---|---|
| Normal | Anosmic | Normal | Anosmic |
| 0.406 | 0.275 | 0.574 | 0.116 |

Expected proportions of OD in sample adjusted by NHANES age-bracketed
proportions

| Normal | Anosmic | Normal | Anosmic |
|---|---|---|---|
| 0.782 | 0.0696 | 0.782 | 0.069 |

The patients reported here were from a symptomatic respiratory clinic population with a median age of 55 years, rather than from a normal healthy population sample used to develop the NHANES data. Respiratory clinic patients over the age of 40 that tested negative for a SARS-CoV-2 infection displayed an incidence of anosmia 1.65 times higher than expected in a normal population, but patients positive for SARS-CoV-2 infection had an incidence ˜3.9 times higher than expected.

This simple ratio of risks is 2.39, consistent with the previous relative risk estimate (2.4) before age correction.

These findings demonstrate that the inventive test kits, such as the paired crushable ampoules used in the study reported in this Example, can be used to detect COVID-19 associated anosmia in clinical settings

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for determining rapidly whether a subject has anosmia and, optionally, whether said subject has hyposmia, said method comprising:
(a) providing said subject with a set of a closed first container and a closed second container, wherein said first container contains a first solution consisting essentially of an odorant at a first selected concentration of said odorant and a carrier and said second container contains a second solution which second solution consists essentially of said odorant at a second selected concentration of said odorant, which selected second concentration is expected to be detectable by all individuals having a normal sense of smell, and said carrier, and further wherein said first container and said second container are either (i) attached to each other, (ii) encapsulated within a common cover member permeable to odors and which is both unreactive with, and which does not comprise a material reactive with, said first solution or said second solution, or (iii) are blisters on a blister pack, and said first and said second containers do not mix when said first and said second containers are opened,
(b) having said subject
(i) open said closed first container, thereby releasing into ambient air around said opened container any odor from said odorant in said first solution in said opened container, and
(ii) smell said ambient air around said opened container, allowing said subject to determine if said subject can smell said odorant in said ambient air, and
(iii) repeat steps (b)(i) and (b)(ii) for said second container,
wherein an inability of said subject to smell said odorant in said second container indicates said subject has anosmia.

2. The method of claim 1, wherein said first selected concentration of said odorant in said first solution is 1 z-score unit below the mean minimum detectable concentration that people with normal olfaction can smell, wherein detection by said subject of said odorant in said second container but not of said odorant in said first solution indicates that said individual has hyposmia.

3. The method of claim 1, wherein said first selected concentration of said odorant in said first solution is 1 z-score unit below the mean minimum detectable concentration that individuals with normal olfaction in a population can smell and wherein said second selected concentration of said odorant in said second solution is a concentration that is expected to be detectable by all individuals having a normal sense of smell and that does not stimulate a trigeminal nerve of an individual in said population, wherein a failure by said individual to detect said odorant both in said first container and in said second container indicates that said individual has anosmia.

4. The method of claim 1, wherein said first selected concentration of said odorant in said first solution is 1 z-score unit below the mean minimum detectable concentration that people with normal olfaction can smell and wherein said second selected concentration of said odorant in said second solution is a concentration that is expected to be detectable by all individuals having a normal sense of smell and that does not stimulate a trigeminal nerve of individuals in said population, wherein detection by said individual of said odorant in both said first container and in said second container indicates that said individual does not have hyposmia or anosmia.

5. The method of claim 1, in which said odorant is n-butanol, and said n-butanol is present in said first container at a concentration of 0.32%±one half log unit and is present in said second container at a concentration of about 1.8%, ±0.2%, to 50%.

6. The method of claim 5, in which said odorant is n-butanol, and said odorant is present in said first container at a concentration of 0.32%, and is present in said second container at a concentration of 3.2%±0.5%.

7. The method of claim 1, in which said odorant is phenyl ethyl alcohol (“PEA”) and said PEA is present in said first container at a concentration of 0.32%, ±one half log unit, and is present in said second container at a concentration of 1.8%, ±0.2%, to 18%.

8. The method of claim 7, in which said odorant is n-butanol, and said odorant is present in said first container at a concentration of 0.32%, and is present in said second container at a concentration of 3.2%±0.5%.

9. The method of claim 1, wherein said first container, said second container, or both, are coded by color, numbering, lettering, or a combination of these.

10. The method of claim 1, wherein said closed first container, said closed second container, or both, are ampoules, blister, or are impermeable packets.

11. A kit for determining rapidly whether a subject has hyposmia or anosmia, said kit consisting of:
a first container and a second container, and,
wherein said first container contains a first solution consisting essentially of a selected odorant at a first concentration and a selected carrier,
and said second container contains a second solution which second solution consists essentially of said odorant at a second concentration and said selected carrier,
further wherein
said first container and said second container are (i) attached to each other, (ii) encapsulated within a common cover member permeable to odors and which is both unreactive with, and which does not comprise a material reactive with, said first solution or said second solution, (iii) are blisters on a blister pack or are containers contained within blisters, or are impermeable packets.

12. The kit of claim 11, wherein said first container, said second container, or both, is an ampoule.

13. The kit of claim 12, wherein said ampoule is, or said ampoules are, encapsulated within a cover member.

14. The kit of claim 11, wherein said first selected concentration of said odorant in said first solution is 1 z-score unit above the mean, 1-tailed, determined in a population of individuals with normal olfaction to be detectable by said population of individuals with normal olfaction and wherein said second selected concentration of said odorant in said second solution is a suprathreshold concentration that does not stimulate a trigeminal nerve of individuals in said population of individuals with normal olfaction.

16. The kit of claim 11, in which said odorant is n-butanol and wherein said odorant is present in said first container at a concentration of 0.32%±one log unit and said odorant is present in said second container at a concentration of about 1.8%±0.2% to 50%.

17. The kit of claim 16, in which said n-butanol present in said first container at a concentration of 0.32% and is present in said second container at a concentration of 3.2%.

18. The kit of claim 11, in which said odorant is phenyl ethyl alcohol (“PEA”) and wherein said odorant is present in said first container at a concentration of 0.32%±one log unit and said odorant is present in said second container at a concentration of about 1.8%±0.2% to 18%.

19. The kit of claim 18, in which said PEA present in said first container at a concentration of 0.32% and is present in said second container at a concentration of 3.2%.
